(19) United States
(12) Patent Application Publication
(10) Pub. No.: US 2009/0220407 A1
(43) Pub. Date: Sep. 3, 2009

(54) PREPARATION AND FUNCTIONALIZATION OF CARBON NANO-ONIONS

(76) Inventors: Luis Echegoyen, Arlington, VA (US); Amit J. Palkar, Clemson, SC (US); Arno S. Rettenbacher, Voels (AT); Frederic Melin, Strasbourg (FR); Bevan Elliot, Greenville, SC (US)

Correspondence Address: DORITY & MANNING, P.A., POST OFFICE BOX 1449, GREENVILLE, SC, 29602-1449, US

(21) Appl. No.: 11/996,611

(22) PCT Filed: Aug. 15, 2006
(86) PCT No.: PCT/US06/31871

§ 371 Date: Sep. 24, 2008

Related U.S. Application Data
(60) Provisional application No. 60/708,148, filed on Aug. 15, 2005.

(51) Int. Cl. C01B 31/02; C07D 207/00; C07C 43/10; C25B 1/00

(52) U.S. Cl. 423/445 B; 548/400; 568/623; 205/555

(57) ABSTRACT

Disclosed are methods for forming carbon nano-onions. The methods include annealing a carbon nanodiamond starting material in an inert atmosphere. The method can be carried out at ambient pressure. Also disclosed are methods for functionalizing carbon nano-onions. For instance, carbon nano-onions can be functionalized so as to be soluble in aqueous or organic solvents, as desired. Also disclosed are methods for separating mixtures of carbon nano-onions. In particular, mixtures of carbon nano-onions can be separated from one another based upon differences in electrochemical characteristics of the different nano-onions.

### RELATED APPLICATIONS

This application claims priority to U.S. Provisional Application Ser. No. 60/708,148, which was filed on Aug. 15, 2005.

### BACKGROUND OF THE INVENTION

The discovery of fullerenes is often considered the beginning of the current nanoscience revolution. During the initial stages of fullerene research, it was discovered that carbon nanostructures, when placed under high energy electron beam irradiation during transmission electron microscopy (TEM) analysis could transform into multi-layer fullerenes, more commonly termed carbon nano-onions (CNOs).

CNO structures were detected as early as 1980, but they remained largely uncharacterized for years, primarily due to the expensive, impractical preparation processes. Recent advances have demonstrated improved preparation methods, such as high temperature annealing of nanodiamond particles under vacuum, implantation of carbon atoms on silver particles, and arcing of graphite underwater. These newer methods still present difficulties due to, for example, associated costs, low yields, and lack of control of the sizes of the CNOs produced, with most methods producing low yields of mixtures of differently sized CNOs.

Though CNOs possess potential suitability for many applications, the realization of this potential has remained elusive due to, for instance, cost-prohibitive formation methods, particularly in regard to high-purity formation methods, a lack of methodology for controlling the size of CNOs produced, and a lack of methodology for the production of functionalized CNOs, which could provide a route to control the characteristics of CNOs. The capability of producing CNOs including predetermined functionalization could be utilized to, for example, improve solubility and dispersibility of the materials in aqueous or organic solvents.

### SUMMARY

In one aspect, the present invention is directed to a method for forming CNOs. For instance, the method can include providing a carbon nanodiamond starting material on a substrate, locating the starting material and the substrate in an inert atmosphere at a pressure of at least about 5 pounds per square inch (psi), heating the inert atmosphere to a temperature of between about 1000° C. and about 2750° C., and then cooling the inert atmosphere at a cooling rate of between about 10° C. and about 30° C. per minute. The disclosed method can form high purity, low defect carbon nano-onions with high yields.

In another aspect, the present invention is directed to methods for functionalizing CNOs and the functionalized CNOs that can be formed by such methods. For instance, the functionalized CNOs can include a polymeric or an oligomeric functional group bound to the outer shell of the carbon nano-onion. Functionalization methods can include, for example, addition reaction via carboxyl groups at the surface of the CNOs, 1-3 dipolar cycloaddition, cyclopropanation using the bromo derivative of diethyl malonate in the presence of a base (the Bingel reaction), and, in the particular case of CNOs formed via an annealing process, free radical addition.

In yet another aspect, the invention is directed to a method for separating a mixture of carbon nano-onions according to differences in electrochemical characteristics of the CNOs in the mixture. In one embodiment, the method can include suspending a mixture of CNOs in a solvent and reducing a first portion of the CNOs until they dissolve in the solvent. According to this embodiment, a second portion of the CNOs can remain in suspension upon the dissolution of the first portion.

In another embodiment, all of the CNOs can be dissolved in a suitable solvent, and a first portion can be selectively deposited on an electrode, leaving a second portion of the CNOs in solution.

### BRIEF DESCRIPTION OF THE DRAWINGS

A full and enabling disclosure of the present invention, including the best mode thereof, to one of ordinary skill in the art, is set forth more particularly in the remainder of the specification, including reference to the accompanying figures, in which:

FIG. 1 illustrates the electron paramagnetic resonance (EPR) data obtained from CNOs formed according to an annealing method as herein described;

FIG. 2 illustrates the difference in aqueous dispersibility following oxidation for CNOs formed according to a graphite arcing method with those formed according to an annealing method as herein described;

FIG. 3 illustrates the CV curve of octadecylamine (ODA)-functionalized CNOs obtained from nanodiamonds according to an annealing method as herein described;

FIGS. 4A and 4B are TEM images of CNOs formed according to a graphite arcing method;

FIGS. 5A and 5B are TEM images of commercially available nanodiamonds;

FIGS. 6A and 6B are TEM images of CNOs obtained from nanodiamonds according to an annealing method as herein described;

FIGS. 7A and 7B are TEM images of CNOs obtained from nanodiamonds according to another annealing method as herein described;

FIGS. 8A and 8B illustrate X-ray powder diffraction results for CNOs formed according to a graphite arcing method (FIG. 8A) and CNOs formed according to an annealing method as herein described (FIG. 8B);

FIG. 9 compares the thermal gravimetric analysis (TGA) of CNOs formed according to a graphite arcing method with those formed according to an annealing method as herein described;

FIG. 10 compares the TGA following oxidation of CNOs formed according to a graphite arcing method with that of CNOs formed according an annealing method as herein described;

FIG. 11 compares the TGA for CNOs formed according to an annealing method as herein described as formed, following oxidation, and following functionalization;

FIGS. 12A and 12B are the ¹H NMR of a polyethylene glycol (PEG) starting material (FIG. 12A) and PEG functionalized CNOs formed according to a graphite arcing method (FIG. 12B);

FIGS. 13A and 13B are the ¹³C NMR of a polyethylene glycol (PEG) starting material (FIG. 13A) and PEG functionalized CNOs formed according to a graphite arcing method (FIG. 13B)

FIGS. 14A-14C are the ¹H NMR of a 1-octadecylamine (ODA) starting material (FIG. 14A), ODA functionalized CNOs formed according to a graphite arcing method (FIG. 14B), and ODA functionalized CNOs formed according to an annealing method as herein described (FIG. 14C);

FIGS. 15A-15C are the ¹³C NMR of a 1-octadecylamine (ODA) starting material (FIG. 15A), ODA-functionalized CNOs formed according to a graphite arcing method (FIG. 15B), and ODA functionalized CNOs formed according to an annealing method as herein described (FIG. 15C); and

FIG. 16 is a flow chart illustrating one embodiment of a CNO functionalization process as herein described.

### DETAILED DESCRIPTION OF THE INVENTION

Reference will now be made in detail to various embodiments of the invention, one or more examples of which are set forth below. Each embodiment is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used in another embodiment to yield a still further embodiment.

In one embodiment, the invention is directed to methods for forming CNOs. Beneficially, the disclosed methods require less in the way of processing equipment and materials and can be carried out at ambient or positive pressure. Accordingly, the disclosed methods can provide many economic benefits as compared to previously known CNO formation methods. In addition, the methods can provide high purity CNOs, with formation of fewer by-products, e.g., nanorods, nanotubes, amorphous graphite, and the like, than many previously known CNO formation methods.

Methods disclosed herein can also be utilized to provide CNOs within a predetermined size distribution range. In one embodiment the CNO formation methods can form CNOs exclusively within the desired range. For instance, methods of the invention can be utilized to form exclusively small CNOs, for example CNOs less than about 10 nm in diameter. In other embodiments, however, exclusively large CNOs can be formed, for instance greater than about 20 nm in diameter.

Separation methods are also disclosed that can be utilized to separate a mixture of CNOs according to differences in electrochemistry of the materials and thereby provide extremely high purity product CNOs.

In another embodiment, the invention is directed to methods for functionalizing CNOs as well as the functionalized CNOs that can be formed according to the methods. For instance CNOs of the invention can be highly dispersible in a liquid, e.g., water. In other embodiments, CNOs can be functionalized with oligomers or polymers exhibiting desired characteristics. For instance, CNOs functionalized as herein disclosed can be soluble.

### CNO Formation

Formation methods according to the present invention can include thermal processing of a nanodiamond starting material. Beneficially, the disclosed process need not be carried out in vacuum conditions, and can also be carried out at temperatures lower than those of many other high-temperature formation methods.

According to one embodiment of the process, nanodiamond can be annealed in an inert atmosphere at ambient or positive pressure. For example, the process can be carried out at a pressure of at least about 5 psi. In one embodiment, the process can occur at a pressure of between about 10 psi and about 20 psi. In another embodiment, the process can be carried out at a positive pressure, for instance greater than about 15 psi. A positive pressure may be preferred in some embodiments, as this may ensure the purity of the atmosphere during the process, and prevent the presence of contaminants in the furnace during the conversion process. In another embodiment, however, the process can be carried out at relatively low pressure, for instance less than atmospheric pressure, for example, between about 1 psi and about 5 psi.

Generally, a nanodiamond starting material can be located in a furnace while held on a substrate. Any suitable thermally stable substrate can be used. In one embodiment, discussed in more detail below, a graphite substrate can be used.

Following placement of a nanodiamond starting material in a furnace the furnace can be purged of air and an inert gas can be provided to the system according to any suitable process. The preferred atmosphere content can vary depending upon the particular characteristics of the system, associated costs, and the like. For example, in those embodiments in which the furnace includes porous components, it may be preferred to utilize a helium atmosphere, so as to ensure complete displacement of any other gaseous materials from the pores of the porous components.

Subsequent to establishment of the inert atmosphere, the furnace interior can be heated to a reaction temperature, for instance a temperature between about 1000° C. and about 2750° C. In one embodiment, the furnace can be heated to a temperature of between about 1200° C. and about 1800° C., for instance between about 1500° C. and about 1700° C.

The furnace can be held at a high temperature for a period of time followed by a slow cooling. For example, the furnace can be held at a high temperature for a period of time between about 30 minutes and about 3 hours, for instance between about 1 hour and about 2 hours.

Following a period of high temperature, the furnace and contents can be slowly cooled. For example, the furnace can be cooled over a period of time of at least about one hour, e.g., at a rate of between about 10° C./minute and about 30° C./minute. The slow cooling can provide substantial time for the materials to reorganize and form the product CNOs. As such, CNOs formed according to the process can be extremely stable. For example, CNOs formed according to the present process can be more resistant to thermal decomposition as compared to CNOs formed according to previously known methods, such as graphite arcing methods, as described in more detail in the Examples, below.

The high temperature annealing process can provide CNOs in high yield. Many previously utilized formation processes, such as graphite arcing, for example, provide a product mixture including CNO, nanotubes, nanorods, amorphous carbon, and the like. According to one embodiment, CNOs can be produced with a product yield of greater than about 90% by weight of the starting material, or even greater in other embodiments, for instance greater than about 95%, or greater than about 98%.

Following formation, the CNO sample can be further processed, if desired. For instance, the CNO sample can be annealed in air to a temperature of between about 300° C. and about 600° C., for instance about 400° C. Annealing at a relatively low temperature can remove byproducts such as amorphous carbon that may be present following the high temperature annealing.

In addition to forming CNOs in high yield, the disclosed process can also be utilized to form CNOs within a predetermined size distribution range. More specifically, the size of the CNOs formed can be controlled through selection of the shape of the substrate used to hold the carbon materials during the process. For example, when the nanodiamonds are held within a container during the process, for instance within a graphite crucible or boat, i.e., a container that includes both a base and walls surrounding the base, the process can form small CNOs, with the majority of the CNOs formed less than about 20 nm in diameter. For instance, at least about 90% by weight of the CNOs formed can be less than 15 nm in diameter. In one embodiment, at least about 85% of the CNOs formed can be less than about 10 nm in diameter. The formation of small CNOs can be beneficial in certain embodiments of the invention, for instance in certain functionalization processes, such as those described below. Smaller CNOs have a higher degree of surface curvature, and are believed to exhibit higher reactivity as compared to larger CNOs, because the stability of the sp² carbon sheet decreases with increased curvature of the sheet.

In another embodiment, large CNOs can be formed by the process. According to this embodiment, the formation process can utilize a flat, or planar, substrate, for example a graphite sheet. According to one particular embodiment, the process can utilize a graphite sheet substrate, and at least about 90% of the CNOs formed can be greater than about 15 nm in diameter. For example, at least about 80% of the CNOs can be between about 20 and about 30 nm in diameter. Larger CNOs may be preferred in embodiments such as those directed to lubrication applications, as they tend to show less tendency toward agglomeration as formed.

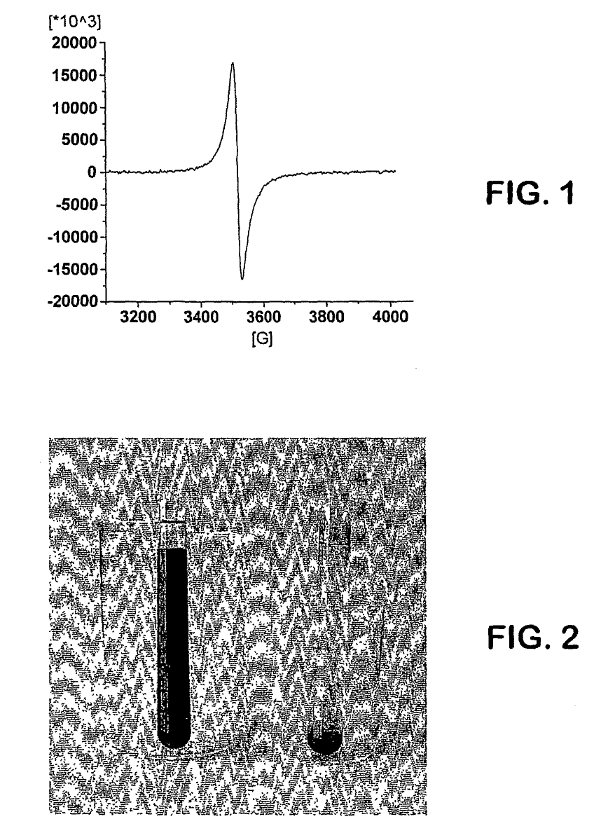

In addition to forming CNOs in high yields, the above-described process can also form CNOs with previously unrecognized characteristics. For example, the CNOs formed according to the above-described process can exhibit unique electronic characteristics as compared to CNOs formed according to many previously know processes, such as underwater graphite-arcing processes. For instance, FIG. 1 illustrates the electron paramagnetic resonance (EPR) data obtained from CNOs formed according to an annealing method as herein described (details of the procedure are described below in the Example section). No EPR signal could be obtained from CNOs formed according to an underwater graphite arcing method such as is generally known in the art. While not wishing to be bound by any particular theory, the electronic characteristics of the CNOs formed according to the disclosed methods are thought to be due at least in part to fewer defects being formed in the graphite sheets of the onions.

In addition, as the disclosed process includes formation in an inert atmosphere, the surface of CNOs formed as described above can include little or no surface hydrogen and thus can exhibit high reactivity to oxidation. Upon oxidation, for instance chemical oxidation via acid treatment, the CNOs formed according to the disclosed process can exhibit excellent dispersibility characteristics. FIG. 2 illustrates the difference in aqueous dispersibility of CNOs formed according to an annealing method in an inert atmosphere as described above followed by reflux for 48 hours with 3.0M nitric acid (shown on the left in the photograph). On the right of FIG. 2 is shown a CNO preparation formed according to a standard underwater graphite arcing method followed an acid treatment and the same dispersal process. As can be seen, the acid treated CNOs formed as described herein are much more dispersible in water.

### CNO Functionalization

According to another aspect, the present invention is directed to organic functionalization of CNOs formed according to any desired method. In particular, while the CNO functionalization regimes described herein are well-suited to CNOs formed according to the annealing methods described above, they are equally suited for CNOs formed according to any other method, e.g., underwater graphite arcing methods, vacuum annealing methods, and the like.

In general, the functionalization regimes described herein employ two basic approaches: direct sidewall addition and functionalization via oxidation of the as-formed CNOs. For example, the latter method can take advantage of carboxylic acid moieties that can be added to the CNO surface via a chemical oxidation treatment, such as that described above. In particular, the carboxyl groups can be utilized according to known addition chemistry to link polymeric or oligomeric groups directly to the CNO surface via, for example, direct acid-base interaction, amidation via the in situ generated acid chloride, or carbodiimide-activated coupling.

For example, according to a direct amidation reaction scheme, following oxidation of the CNOs, for instance chemical oxidation via acid treatment, CNOs can be functionalized with a desired oligomer or polymer according to the following reaction:

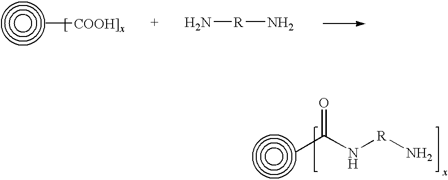

wherein R can be any suitable group such as, for example a poly(ethylene glycol) of any desired length, an alkyl chain (CH2)n, an aromatic group, and the like.

Reaction conditions can vary according to specific materials, desired yields, etc. as is generally known in the art. For example, when functionalizing CNO with a poly(ethylene glycol) (PEG) polymer, higher reaction temperature (up to about 140° C.) and longer reaction times (more than about 6 days) can give higher product yields. When considering other R groups, for instance, straight chain alkyl groups, preferred reaction conditions can vary. Such variations are generally known to those of ordinary skill in the art, and thus are not discussed at length herein.

According to another embodiment, CNOs can be functionalized via a 1,3 dipolar cycloaddition reaction scheme. This functionalization scheme is based on the 1,3-dipolar cycloaddition of azomethine ylides generated by condensation of an α-amino acid and an aldehyde. For example, to improve solubility an alkyl chain can be attached out the outer shells of the CNOs. As the azomethine ylide reaction requires an amino acid and an aldehyde to generate the 1,3-dipolar species for the cycloaddition, it is possible to introduce the alkyl chain via either the aldehyde or the amino acid. One exemplary embodiment of the general reaction scheme is illustrated below:

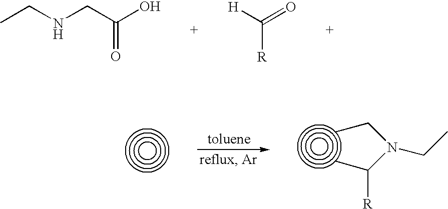

wherein the aldehyde can be provided in excess, for instance about 15 equivalents, and

R can be any desired alkyl chain.

In one preferred embodiment, the chain length can be selected to ensure solubility. For example, an alkyl chain of at least about 12 carbons can be bound to a CNO.

Reaction conditions can vary, as is known in the art. For example, energy can be provided via thermal heating or microwave irradiation, as desired. Utilization of microwave irradiation can facilitate the cycloaddition and lead to better yields in shorter time, though cost considerations may also be a factor in preferred energy source.

According to another functionalization scheme, a predetermined polymer or oligomer can be bound to a CNO via a Bingel reaction. The Bingel reaction has been found efficient in the past in the cyclopropanation of fullerenes. For example, CNOs can be functionalized according to Bingel reactions similar to those described for fullerenes by Camps, et al. (J. Chem. Soc., Perkin Trans. 1, 1997, p. 1595), which is incorporated herein by reference.

In general, this particular embodiment is directed to the cyclopropanation of CNO via reaction with bromomalonates in the presence of a base. This functionalization method can be preferred in some embodiments of the invention due to the mild reaction conditions that can provide relatively high yields, the exclusive formation of [6,6]-bridged adducts, and relatively simple, one step access to higher adducts (bis up to hexakis) with a stereochemically defined addition pattern, for instance using template activation with 9,10-dimethylanthracene (DMA).

In one embodiment, the method can be carried out through utilization of the bromo derivative of diethyl malonate in the presence of a base such as sodium hydride or diazobicyclo[5.4.0]undec-7-ene (DBU). A modification of this embodiment, in which the CNO is directly treated with diethyl malonate in the presence of iodine and base, can optionally be utilized with relatively high yields. In another embodiment, good yields can be achieved by direct treatment of the CNO with malonates in the presence of CBr₄ and DBU.

In yet another embodiment, a mixture of malonate, bromo-malonate, and dibromo-malonate can be used as the reagent for the cyclopropanation reaction, as the bromo-malonate is the only reactive species of this mixture.

According to yet another embodiment, CNOs formed according to a high temperature annealing process, such as that described above, can be functionalized via free radical reactions. For instance, reactive aryl radicals, e.g., phenyl radicals, that are able to covalently bind CNO according to the following reaction scheme can be utilized to functionalized CNO (in this reaction scheme, the CNO wall is typified by the vertical line).

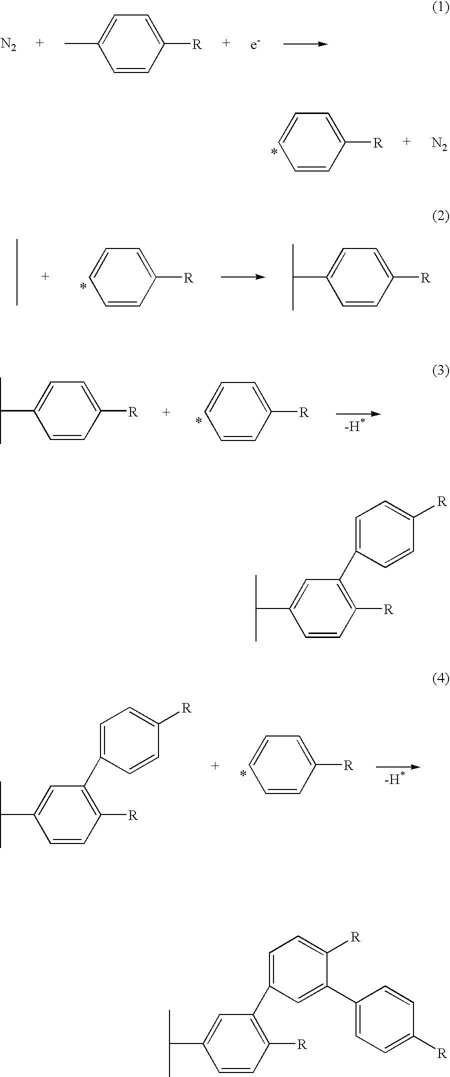

wherein R is any desired functional group and

the aryl group can be phenyl, naphthyl, anthraquinone, or the like.

The initiating reaction is electron transfer to the diazonium salt to generate the reactive phenyl radical and liberate nitrogen gas, as depicted schematically in reaction (1) above. The radicals then react covalently with the CNO surface and can form the first monolayer film, as shown in reaction (2) above. It is believed that electron transfer through this film can be very efficient, and that production of the phenyl radicals can continue as long as the electrolysis is maintained and as long as there is diazonium salt present in solution. The reactive phenyl radicals are able to further derivatize the monolayer and the film thickness can increase as the reaction proceeds, as depicted in reactions (3) and (4) in the scheme above. Thus, this particular functionalization scheme may be particularly attractive for development of an encapsulating film at the CNO surface.

Aryl radicals can be generated according to either electrochemical reductions methods or chemical reductions, as is generally known in the art. For instance, an in-situ method of diazonium salt generation (e.g., aniline with isoamyl nitrite) can be utilized.

Alternatively, electrochemical methods can be used to generate the diazonium salt. For example, CNOs can be deposited on surface supports that can be used as working electrodes. In one embodiment, platinum mesh electrodes can be coated with CNO suspensions followed by solvent evaporation.

In another embodiment, CNOs can be deposited on Teflon™ membranes or some other similar porous and inert support via filtration from organic solvent suspensions. Relatively thin films may be preferred in order to avoid the problem of surface effects, but as functionalization can lead to solubilization of the CNOs, continuous removal from the film surface can also be encouraged through selection of the particular R group. Optionally, bucky paper (i.e., bundled carbon nanotubes) can be used as a support, to improve conductivity.

### Electrochemical Separation of CNOs

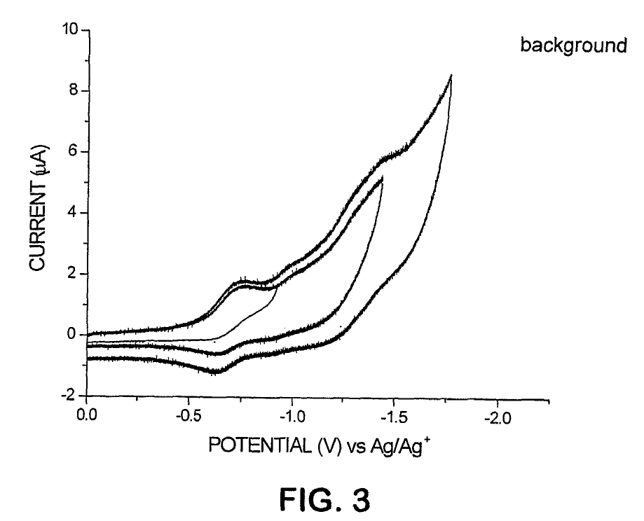

FIG. 3 illustrates the CV curve of octadecylamine (ODA)-functionalized CNOs obtained from nanodiamonds according to an annealing process under an inert atmosphere (specific details of the formation method are described in the Example section, below). As can be seen, the trace includes several well resolved signals. While not wishing to be bound by any particular theory, it is believed that individual waves of the trace are from CNOs of particular sizes. In any case, the figure illustrates that the solution includes a mixture of different CNOs that exhibit different electrochemical characteristics. Accordingly, one embodiment of the present invention is directed to separation of a mixture of CNOs via an electrochemical separation process.

One particular embodiment of an electrochemical separation according to the present invention can be similar to that described by Diener, et al. (U.S. Pat. No. 6,303,016), which is incorporated herein by reference, and describes a method of isolation of small-bandgap fullerenes and endohedral metallofullerenes.

In general, a mixture of CNOs can be separated via controlled reduction of the different CNOs. More specifically, the charge state of certain of the CNOs in a mixture can be altered, leading to a change of physical state for those CNOs (e.g., solid to liquid or liquid to solid, while other CNOs that possess different electrochemical characteristics, can remain in the initial physical state.

For example, in one embodiment, the mixture of CNOs can be provided as suspended solids, and the different CNOs can be separated through selective dissolution based upon the difference in electrochemical characteristics of the different CNOs. To electrochemically reduce the targeted CNOs and render them soluble in this particular embodiment, the mixture of CNOs can be dispersed in an organic solvent. For instance, the CNOs can be dispersed in a benzonitrile solution containing an electrolyte, e.g., 0.1 M tetrabutylammonium hexafluorophosphate (TBA+PF₆—). The solvent is not limited to this, however, and any other suitable organic solvent can be optionally utilized such as, for example, tetrahydrofuran (THF), dichloromethane (CH₂Cl₂), 1-methyl-2-pyrrolidinone, or any other solvent or solvent mixture capable of solubilizing the supporting electrolyte and dispersing CNO anions. Similarly, other electrolytes, such as KPF₆, TMAPF₆ or TBABF₄, and the like can be utilized.

Following formation of the suspension, a predetermined voltage can be applied to a working electrode in the suspension. This potential can be just negative of the voltage required to reduce the first targeted species in the mixed sample. The decay of the rate of charge transfer to the solution can be monitored. While not wishing to be bound by any particular theory, it is believed that the addition of charge during the reduction process can supply the electrons needed to give the targeted CNOs a stable closed shell electronic configuration. This allows the generated anions to dissolve while leaving the other CNOs, those held in a matrix with stronger bonds, suspended in the mixture.

Once reduced, the solution can be filtered according to standard methods to separate the now dissolved CNOs from those remaining in suspension.

Following filtration, the filtrate can be oxidized at positive potentials and the dissolved CNOs can then plate out on the working electrode.

According to an alternative embodiment, a mixture of CNOs can be separated according to their different electrochemical characteristics from solution, rather than from solid. According to this embodiment, as-formed CNOs can be functionalized, for instance according to one of the methods described above, such that the CNOs are soluble in a solvent capable of supporting an electrolyte. The solution can thus include a mixture of functionalized CNOs. A suitable electrolyte can be added to the solution and a predetermined voltage can be applied to a working electrode in the suspension. This potential can be just negative of the voltage required to reduce the first targeted species in the mixed sample and deposit this species on the support, leaving the other CNOs in solution.

As carbon nano-onions can show the ability to accept electrons, it is believed that they may be useful in the construction of electronic devices, such as capacitors, for instance.

Other potential applications for the CNOs described herein can include optical limiting, catalysis, gas storage, and the like. The CNOs also show utility in photovoltaic and fuel cell applications. For instance, CNOs, which possess a much larger surface area than single-walled carbon nanotubes, can be excellent candidates for the development of miniaturized fuel cells. CNOs have also been shown to exhibit excellent tribological properties, and could be utilized in aerospace applications. They can provide adequate and even superior lubrication as compared to the commonly used graphitic materials.

The invention may be further understood with reference to the Examples, below.

### EXAMPLES

### Experimental Procedures

All chemicals were of analytical grade and used as received without further purification. Water was purified by a Milli-Q water purification systems (millipure) to a resistivity of 18.2 MΩ. The copper grids for TEM were bought from TED PELLA, Inc. (Prod. No. 08130, 200 mesh, Silicon Monoxide/formvar).

¹H and ¹³C NMR spectra were recorded on a Jeol Eclipse +500 spectrometer. Spectra were referenced to the residual proton resonance in CDCl₃ (δ=7.26 ppm, δ=7.70 ppm for ¹³C) as the internal standard. Chemical shifts (δ) were reported as parts per million (ppm) on the δ scale downfield from TMS. Assignments were confirmed either by 2D NMR experiments or by means of NMR simulation software (ACD NMR predictor).

Size and morphology of materials were observed using a Hitachi Model (TEM-9500) transmission electron microscope of high resolution images (HRTEM 300 kV, Hitachi Model HD-2000 transmission electron microscope of high resolution images (HRTEM, 200 kV) and a Hitachi Model H7600T transmission electron microscope for low resolution images (TEM, 120 kV). The specimens under investigation were prepared from solution, dispersion, or embedded resin slices and deposited on copper grids.

For the preparation of 20 nm slices with an ultramicrotome (RMC PowerTome-XL), the sample under investigation was embedded in LR White Resin, a polar monomer polyhydroxylated aromatic acrylic resin. Less than 1 mg of compound was added to a vial filled with 1 mL of the resin. After curing the resin by heating overnight at 60° C., thin slices were cut with self prepared glass knives (RMC glass knife maker) with the ultramicrotome (cutting speed 3 mm/s). The 20 nm thin slices were collected in a vessel filled with deionized water, subsequently deposited onto TEM copper grids, and dried in a desiccator overnight before TEM experiments.

TGA was conducted on a Mettler TGA/SDTA851. In a typical experiment, a sample (18.5 mg) was loaded into an alumina pan under constant nitrogen or ambient air flow (50 mLmin-1). The temperature ceiling was set at 500° C., with a relatively slow scanning rate (5° Cmin-1) to ensure a complete thermal defunctionalization (in the case of functionalized CNO). For crude CNO the temperature ceiling was 850° C. and heating rate 20° C./min.

Raman spectra were recorded using a Renishaw 1000 Raman spectrometer with the 785 nm emission line of a near infrared laser as the excitation source.

The experimental details for the electrophoretic deposition of modified and unmodified CNOs as films on electrode surfaces have been described elsewhere by Kamat et al. (J. Phys. Chem. B, 2004, 108, 19960). Tetraoctylammonium bromide (TOAB) was used to disperse non-soluble samples.

### Example 1

### CNO Formation

Graphite arcing experiments were conducted under deionized water (18.2MΩ, as described in the literature, using high purity graphite (POCO grade ZXF-5Q) for both the anode (0.25 inch) and cathode (1.0 inch). Arcing was carried out at 30 Å, as running the arc discharge at lower currents resulted in very unstable arc discharges, and higher currents gave rise to very exothermic and violent reaction conditions. The automated onion producing mechanism utilized a DC gear motor and a variable output DC power supply. The power supply was adjusted to drive the motor at the correct rate of speed to maintain an optimum distance between the electrodes for a constant-voltage arc, enabling a higher synthetic output rate than would be possible with only manual control.

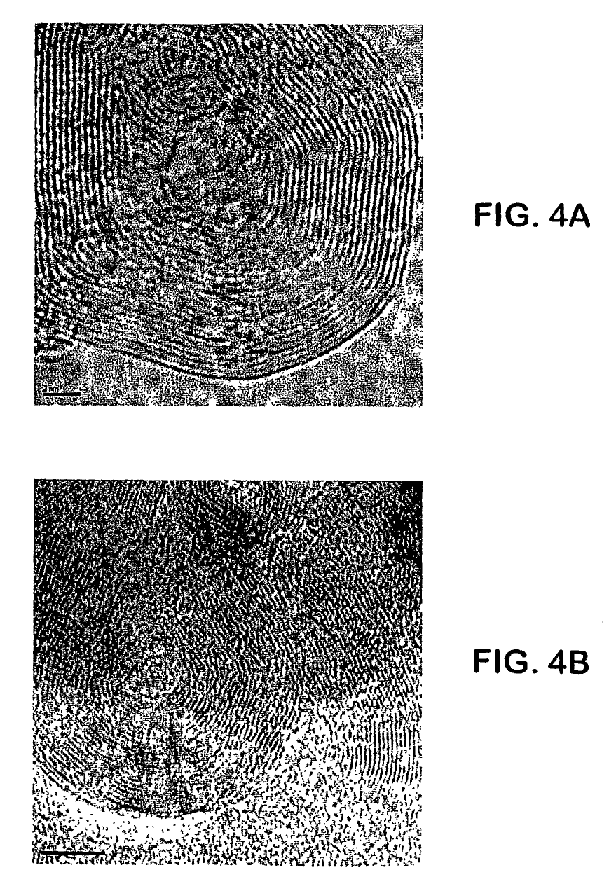

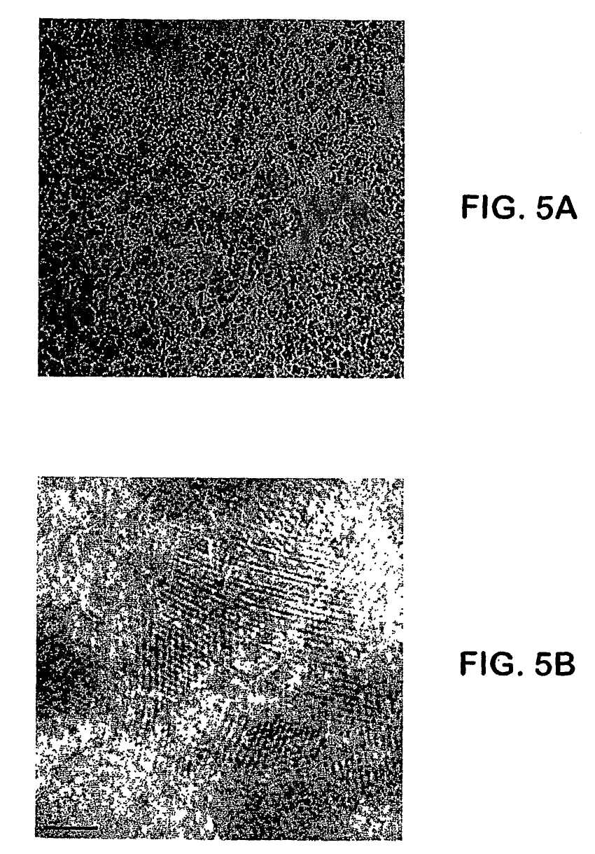

FIGS. 4A and 4B are TEM images of CNOs formed according to this method. Size reference lines on the figures are 2 nm on FIGS. 4A and 5 nm on FIG. 4B. These CNOs were found to be approximately 20 nm in diameter (approximately 30 layers) and clearly showed the proper graphitic interlayer distance of 0.33 nm.

CNOs were also formed according to an annealing process under an inert atmosphere. Approximately 1 gram of pure nanodiamond was placed on one of two different substrates, either a graphite crucible or a graphite sheet. FIGS. 5A and 5B are TEM images of the nanodiamond material used. Size reference lines on the figures are 20 nm on FIGS. 5A and 2 nm on FIG. 5B.

Substrates were transferred to an Astro carbonization furnace. The air in the furnace was removed by applying vacuum followed by purging with helium. The process was repeated twice to ensure complete removal of air. The nanodiamonds were then heated up to 1650° C. under helium atmosphere using a heating ramp of 20° C. per minute. The final temperature was maintained for one hour and then the material was slowly cooled to room temperature over a period of one hour.

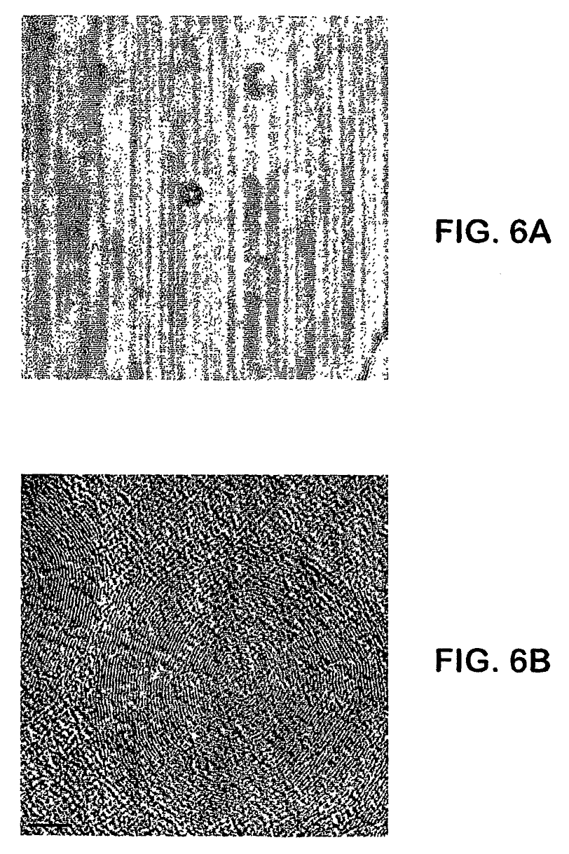

FIGS. 6A and 6B are TEM images of CNOs formed on a graphite sheet. Size reference lines on the figures are 100 nm on FIGS. 6A and 5 nm on FIG. 6B. CNOs obtained by the process were found to be between about 20 nm and about 30 nm in diameter.

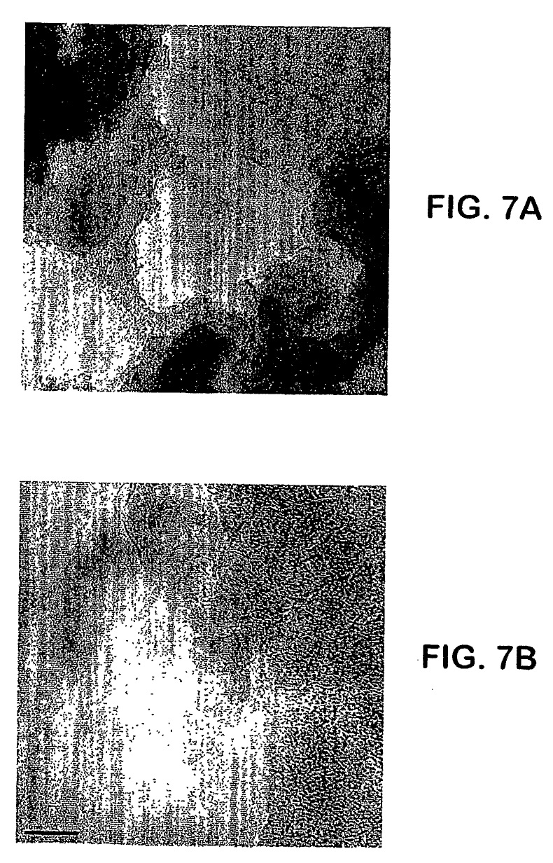

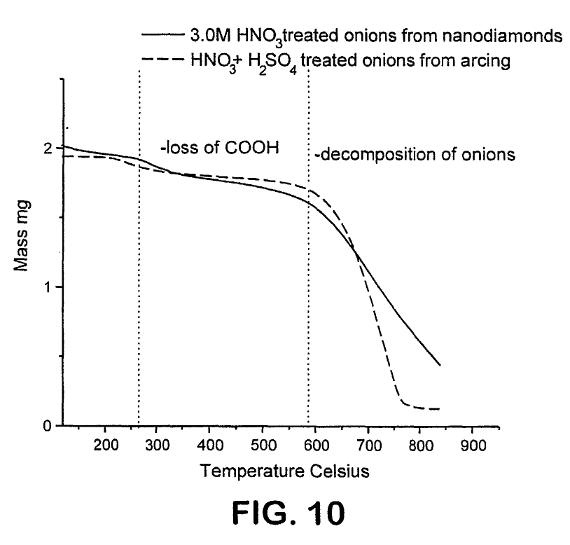

FIGS. 7A and 7B are TEM images of CNOs obtained from nanodiamonds formed in a graphite crucible. Size reference lines on the figures are 100 nm on FIG. 7A and 10 nm on FIG. 7B. Average size of the formed CNOs was about 6 nm. The product was also found to be highly aggregated.

The nano-onions formed according to both methods were annealed in air at 400° C. for one hour to remove any amorphous carbon present.

Following annealing in air, the CNOs formed according to both the arcing and the annealing methods were oxidized under convective heating by refluxing for 48 hours with acid. The CNOs formed from nanodiamond via the annealing method were refluxed with dilute nitric acid (3M). The CNOs formed according to the graphite arcing method did not exhibit high levels of functionalization upon nitric acid treatment. As such, a harsher method of oxidation was employed for the CNOs formed according this method. In particular, the CNOs formed via the arcing method were oxidized utilizing a mixture of concentrated nitric and sulfuric acids.

The CNOs were characterized by EPR as discussed above. CNOs formed from nanodiamond according to the annealing process exhibited the EPR illustrated in FIG. 1, while no EPR response was obtained for the CNOs formed by the graphite arcing process.

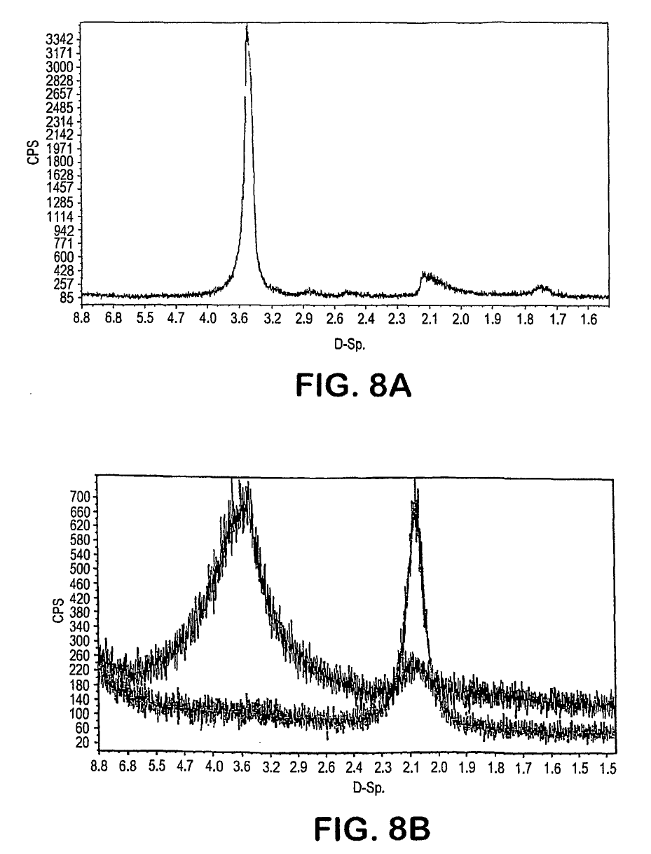

The CNOs were also characterized by X-ray powder diffraction, results of which are shown in FIGS. 8A (CNOs from graphite arcing) and 8B (CNOs from nanodiamond annealing). The lighter, bottom trace on FIG. 8B shows the characteristics of the starting nanodiamond material. As can be seen, both products showed similar results. Similarly, Raman spectra (not shown) of CNOs formed according to both methods showed the characteristics G band and D band of carbon nano-onions reported in the literature.

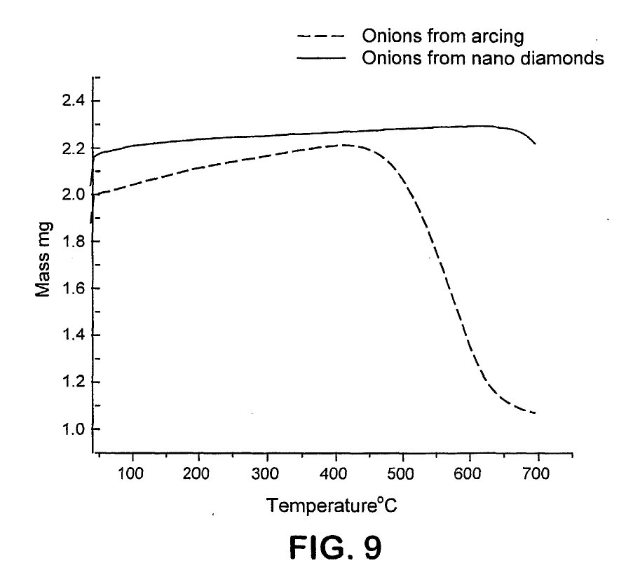

FIG. 9 illustrates TGA of the crude nano-onions from both methods. As can be seen, the CNOs formed according to the graphite arcing method show a lower decomposition temperature, believed to be due to the larger amount of defects formed in the CNOs according to this formation method. Moreover, the CNOs formed according to the annealing process exhibit a decomposition temperature very close to that of C₆₀, i.e., 700° C.

FIG. 10 illustrates TGA of the CNOs from both methods following acid treatment. As can be seen, the materials exhibit similar decomposition characteristics following addition of the carboxyl groups, however, as discussed above, CNOs from arcing require harsher oxidation conditions [HNO₃+H₂SO₄] to show results similar to the CNOs formed from nanodiamonds via the disclosed annealing process.

### Example 2

### Synthesis of PEGylated CNOs

In a typical experiment, annealed and nitric acid-treated (3M, 48 h reflux) CNOs (29.5 mg) formed according to the underwater graphite arcing method described in Example 1 were mixed with PEG₁₅₀₀ₘₙ (474 mg) and stirred for 19 days under argon in a round-bottom flask immersed in an oil bath heated to 140° C.

After cooling to room temperature, deionized water (10 mL) was added to the flask and the resulting black-red suspension was sonicated for 5 minutes and transferred quantitatively to a SpectraPore membrane tubing (molecular weight cutoff ca 12 kD) for dialysis against fresh deionized water for 76 hours under vigorous stirring. The water surrounding the tube was replaced every 24 hours. After one day, the water had a light yellow color, but subsequent water portions remained colorless. After several centrifuging/decanting steps (until the supernatant aqueous solution remained colorless) and heat drying the vacuum oven until the weight became constant, 17 mg of unreacted starting material was recovered. The dark-colored water solution of PEG150 nm functionalized CNOs was evaporated and after drying in the vacuum oven, 184 mg of a red brown viscous oil was obtained.

In order to eliminate unwanted side products, another dialysis was done (SpectraPore membrane tubing, molecular weight cutoff ca. 300 kD). After 24 hours, the outside water became yellow, but remained colorless thereafter. The remaining black solution inside the membrane was evaporated, dried and yielded 15% of the starting material as a black powder, which could be re-dissolved in water or any common organic solvent. The yellow solution outside the tubing was evaporated and yielded the remaining 85% as a red oil after drying in the vacuum oven.

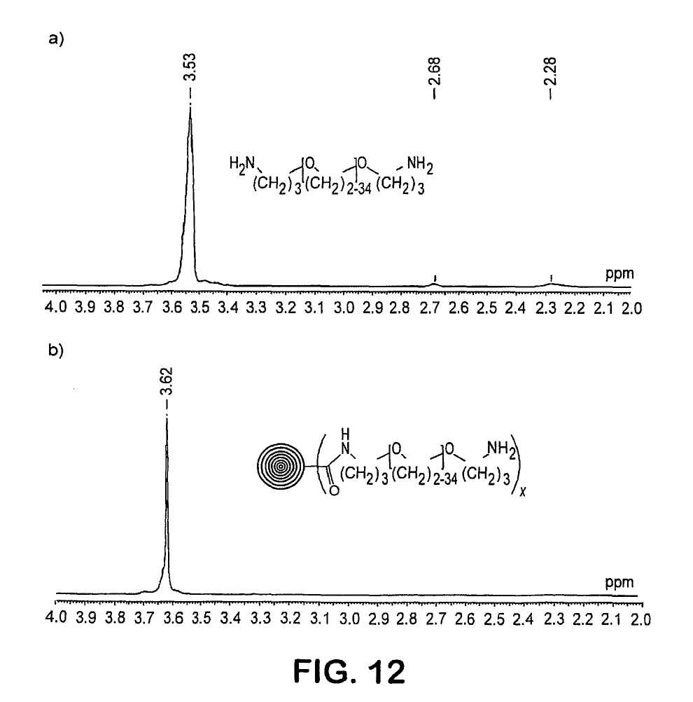

A comparative study of the ¹H NMR spectra of the PEG starting material (FIG. 12A) and the PEGylated CNOs in CDCl₃ (FIG. 12B) shows small but significant differences. In both spectra the very intense multiplet at 3.53 ppm for PEG and 3.62 ppm for PEGylated CNOs dominates the spectrum as expected since this strong signal corresponds to the 68 CH₂ groups vicinal to two oxygen atoms. However, the two weak broad signals at 2.28 and 2.68 ppm (the six terminal CH₂ groups) in the 1H NMR for the starting material disappear completely in the ¹H NMR for the PEGylated CNOs. The absence of signals from the terminal CH₂ groups suggests a successful derivatization of the carboxylated CNOs, at both of the terminal NH₂ groups of the long PEG chain. The closer the protons or carbon atoms of the PEG chain are to the outer shell of the CNO, the more likely they will experience different chemical environments due to the presence of CNOs of different sizes and to the close proximity to other PEG chains. Therefore, the signals from these nonequivalent nuclei do not add up to one strong signal, but are each too weak to be observed.

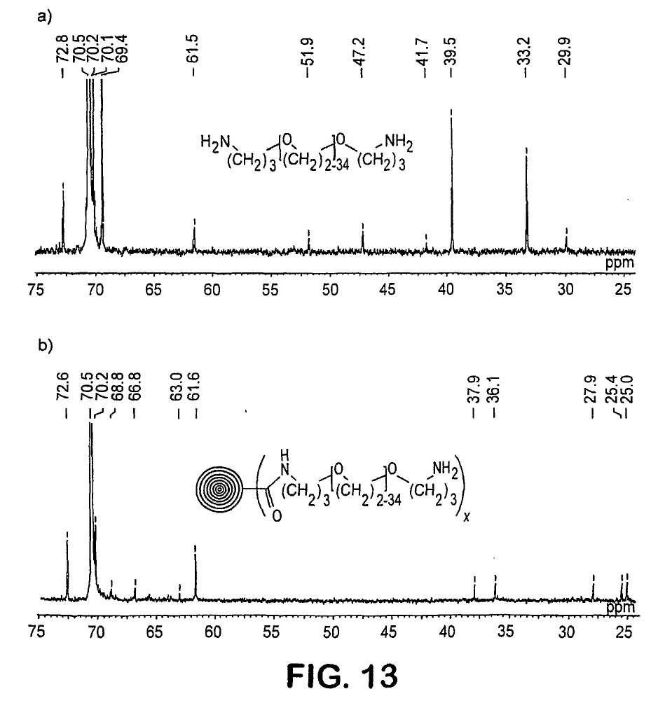

A comparative study of the ¹³C NMR spectra (FIG. 13A, starting PEG, and FIG. 13B, PEGylated CNOs) yields clearer results and shows similar behavior. Some chain carbon signals disappear completely in the functionalized CNO sample, while others remain visible and unshifted. The three strong signals at 33.2 (C-2, where the number refers to the relative position from the terminal hetero element N), 39.5 (C-1), and 69.4 (C-3) pm in the PEG starting material spectrum disappear in the PEGylated CNO spectrum. This again suggests that the signals from at least the three carbon atoms (C-1, C-2, C-3) closest to the CNO cage are broadened beyond observation for the same reasons already mentioned.

### Example 3

### Synthesis of Octadecylated CNOs (Solid-State Reaction)

In a typical experiment, 15.4 mg of the graphite arcing-formed CNOs (annealed and refluxed in HNO3) and 1-octadecylamine (ODA) (50 mg) were placed together into a glass ampoule and evacuated to ca. 0.1 mbar, before the ampoule was sealed. The ampoule was placed in an oven preheated to 170° C. and baked for 2 hours. The ampoule was opened, put into a round bottom flask and the excess 1-octadecylamine was removed by evacuation of the flask with simultaneous heating at 170° C. After cooling to room temperature, 10 mL of a 1:1 mixture of THF/CHCl₃ was added to the flask and the solution ultrasonicated for 1 hour. After several cycles of centrifuging, decanting, adding of new solvent mixture to the black residue, ultrasonification etc., a dark black green transparent solution was obtained, which proved to be stable for months. The black residue, containing non-reacted and only partially derivatized CNOs, weighed 14.6 mg after drying in the vacuum oven (50° C.). The yield of the soluble part was 6.0 mg after evaporation of the solvent and drying in the vacuum oven (50° C.).

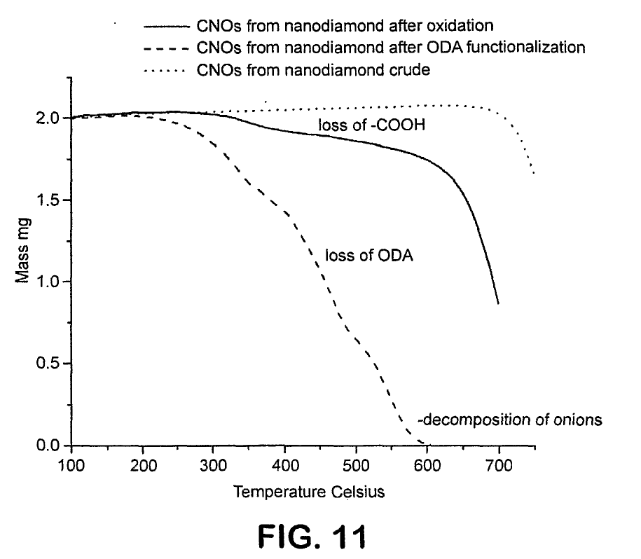

This method was carried out with samples of the CNOs formed on graphite sheets from nanodiamond according to the annealing method as described in Example 1. When utilizing these CNOs, no centrifuging, decanting, or addition of new solvent was necessary. These onions readily dissolved in chloroform and could even be filtered through 0.2 um PTFE membrane. FIG. 11 compares the TGA for these CNOs as formed, following oxidation, and following ODA functionalization formed according to an annealing method as herein described. As can be seen, additional functionalization of the CNOs leads to decreasing decomposition temperature.

The electrochemical characteristics of the ODA functionalized CNOs formed on graphite sheets from nanodiamond according to the annealing method described in Example 1. The conditions were as follows:

working electrode: glassy carbon 3.0 mm diameter

counter electrode: platinum

reference electrode: silver wire

solvent: dichloromethane

electrolyte: 0.1 M tetrabutylammonium hexafluorophosphate

scan rate: 100 mV/s

atmosphere: argon

Results are illustrated in FIG. 3. As can be seen, the scan includes well-resolved signals. The different nano-onions of the mixture can be separated from one another according to electrochemical separation techniques, such as those described above.

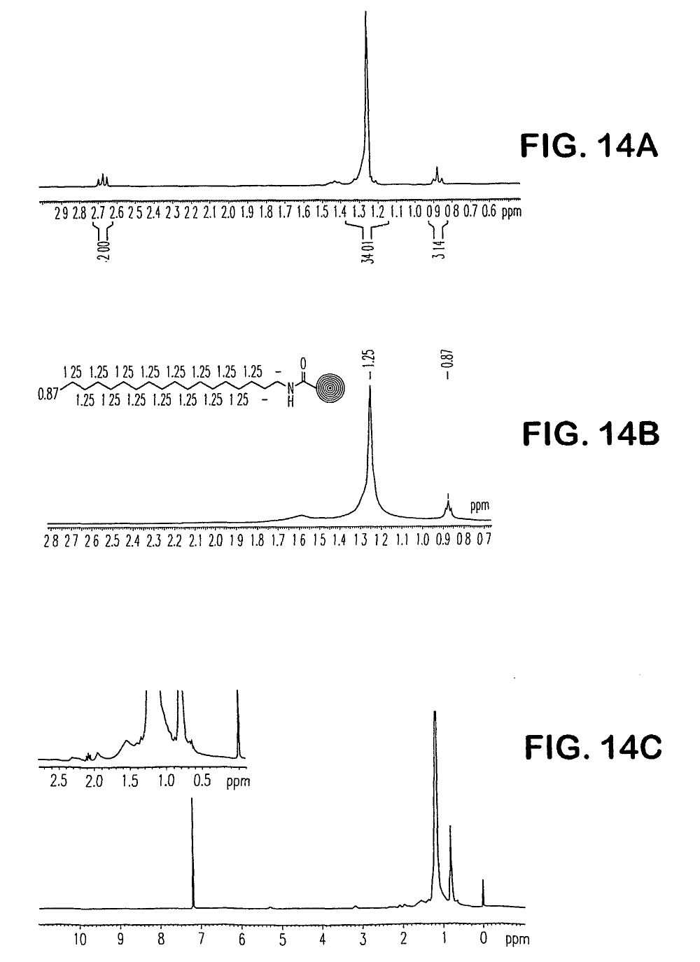

¹H and ¹³C NMR experiments of starting material and CNOs functionalized according to both methods were carried out and compared. FIG. 14A shows a 500 MHz 1H spectrum of the starting ODA, FIG. 14B shows a similar spectrum from the ODA derivatized CNOs formed via graphite arcing, and FIG. 14C shows a similar spectrum from the ODA-derivatized CNOs formed from annealed nanodiamond. The triplet at 2.65 ppm (CH2-1) disappears in the derivatized CNO spectrum, as does the multiplet at 1.40 ppm (CH₂-2). The broad signal around 1.6 ppm in FIG. 14B is due to HOD since addition of D₂O shifted the signal (HOD does not appear in FIG. 14A due to a higher concentration of the ODA). The signals from the CH₃ group at 0.85 ppm (0.87 ppm in the ODA-derivatized CNOs) and the CH₂ groups at 1.23/1.24 ppm did not disappear, because these are farther away from the carbon onion surface.

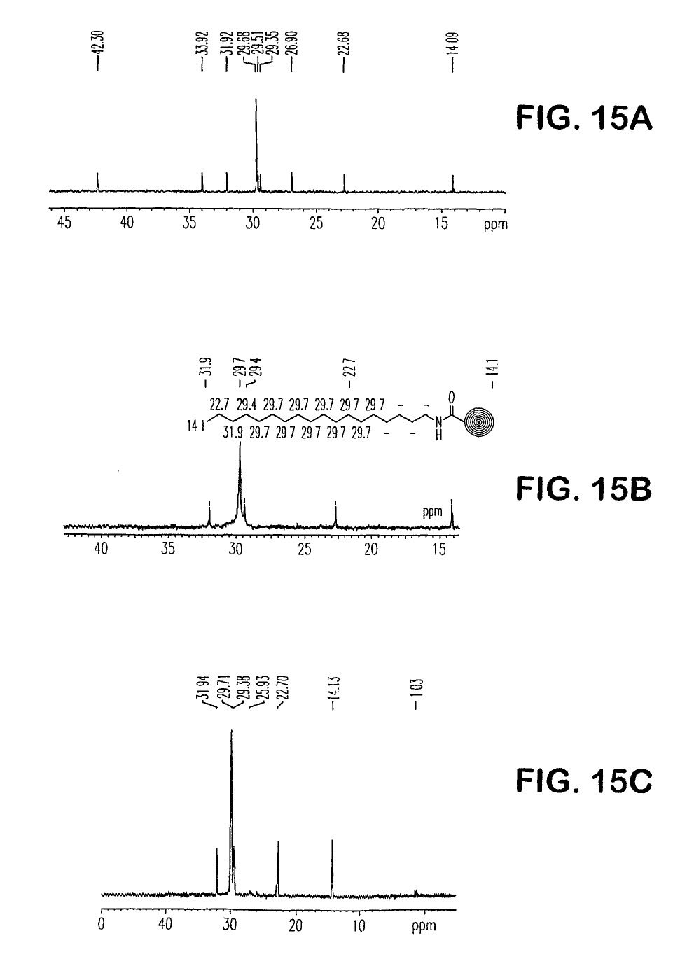

Even more compelling is the comparative ¹³C NMR spectra (FIGS. 15A-15C). The four closest carbon atoms at 42.3 (C-1), 33.9 (C-2), 26.9 (C-3) and 29.5 (C-4) disappear completely in the derivatized CNO spectra (FIGS. 14B, arcing CNOs, and 14C, annealed CNOs).

The very large number of carbon atoms in the outer shell of different-sized CNOs make their individual detection by carbon NMR unlikely. After running a saturated sample of ODA-derivatized CNOs for via the underwater graphite arcing method for 48 hours, a significant rise of the background in the expected aromatic carbon region was visible (maximum at about 115 ppm). A similar experiment with an ODA solution under the same conditions showed no aromatic carbon signal. Therefore, it is probably that this broad signal arose from the collection of nonequivalent carbons in the nano-onions' outer cages.

### Synthesis of Octadecylated CNOs (Microwave Reaction)

In a typical experiment, annealed and acid treated CNOs (19.7 mg) were reacted with ODA (100 mg) and DMF (20 mL) in a 100 mL reaction Teflon chamber under microwave conditions (CEM Model 205 fitted with pressure and temperature controllers). After evaporating the DMF suspension, the excess ODA could be totally removed by heating the residue in vacuum (150° C., 0.1 mbar). To the remaining 27.9 mg black powder, CHCl3 (5 mL) was added and the solution sonicated for 4 h. The dark black green suspension was centrifuged for 2 h, the green transparent supernatant decanted, the black residue treated with another portion of the fresh CHCl3, and the whole procedure repeated until the supernatant remained colorless. Finally 11.5 mg of black powder soluble in CHCl3 (derivatized CNOs) and 16.4 mg of an unsoluble black residue (underivatized and partially derivatized CNOs) were obtained after drying in the vacuum oven (50° C.).

The weight gain due to derivatization by this method was slightly better (42% compared with 39% for the solid-state reaction). The Raman spectrum (not shown) for this product also showed no uncharacteristic features. Comparing the results from the microwave reaction with the solid-state reaction by TEM, the latter reaction yield was slightly less, but was qualitatively similar in all other respects. NMR investigations of this sample (not shown) showed exactly the same results as discussed above for the ODA-derivatized CNOs obtained by the solid-state reaction.

### Example 4

### Pyrrolidine-Derivatized CNOs

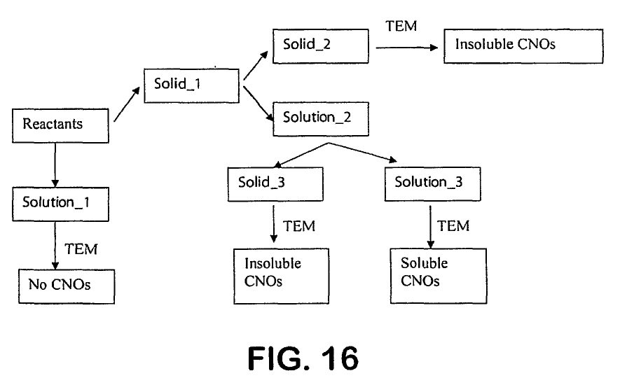

In a typical experiment, annealed and nitric acid-treated (3M, 48 h reflux) CNOs (4 mg) formed according to the underwater graphite arcing method described in Example 1, were annealed again for 1 h at 400° C. The CNOs were combined with N-ethylglycine (2 mg) and dodecanal (68 μL) and sonicated for 15 min in dry toluene (5 mL) and thereafter refluxed under argon for 4 d. After cooling to room temperature, centrifuging of the suspension yielded a black solid_{—}1 plus an orange solution_{—}1 (see the flow chart of FIG. 16). Solid_{—}1 was sonicated with freshly distilled toluene (5 mL) for 5 min. After centrifuging for 10 min, a slightly yellow clear toluene layer with black solid on the bottom was obtained. The toluene phase was added to solution_{—}1. After a second addition of fresh toluene and another centrifuge cycle, a totally colorless toluene phase was obtained which again was added to solution_{—}1. The vial with the collected toluene solutions was set aside (a TEM of this solution did not give any evidence of CNOs). Vacuum oven drying (50° C.) of solid_{—}1 afforded a black powder (4.7 mg), which was sonicated in CHCl₃ (5 mL) for 1 h. After centrifuging for 1 h, a deep black green solution_{—}2 was obtained with black solid on the bottom. After decanting solution_{—}2 and setting it aside, another 5 mL CHCl₃ were added, and the entire procedure repeated until the supernatant CHCl₃ solution remained colorless. All CHCl₃ solutions were collected, evaporated to dryness and vacuum dried at 50°, to afford a black solid-3 (0.5 mg). The remaining, insoluble black solid_{—}2 weighed 4.2 mg after vacuum drying.

Following the same procedure as described above with the homologous aldehyde tridecanal under similar conditions (longer reaction time of additional 2.5 d) resulted in higher yields: starting from annealed CNOs (33.0 mg), a THF soluble black powder (18.6 mg) was obtained, as well as insoluble material (30.0 mg).

The pyrrolidine-derivatized CNOs were electrophoretically deposited on ITO electrodes to obtain Raman spectra of good quality. The spectra (not shown) had a sharp G band at 1580 cm-1, the so-called D′ band at 1609 cm-1 as a shoulder and the D band at 1311 cm-1. The pyrrolidine-derivatized CNOs also exhibited a strong band at 2618 cm-1.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole and in part. Furthermore, those of ordinary skill in the art will appreciate that the foregoing description is by way of example only and is not intended to limit the disclosure so further described in such appended claims

What is claimed is:

1. A method for forming carbon nano-onions comprising:
providing carbon nanodiamond starting material to a substrate;
locating the starting material in an inert atmosphere at a pressure of at least about 5 pounds per square inch;
heating the inert atmosphere to a temperature between about 1000° C. and about 2750° C. while the starting material held on the substrate is in the inert atmosphere; and
following the heating step, cooling the inert atmosphere to ambient temperature at a cooling rate of between about 10° C. and about 30° C. per minute;
wherein the method forms carbon nano-onions from the carbon nanodiamond starting material.

2. The method according to claim 1, further comprising annealing the product carbon nano-onions in air at a temperature of between about 300° C. and about 400° C.

3. The method according to claim 1, wherein the substrate is a graphite substrate.

4. The method according to claim 3, wherein the substrate is a graphite crucible.

5. The method according to claim 3, wherein the substrate is a graphite sheet.

6. The method according to claim 1, wherein the substrate comprises a base and walls surrounding the base.

7. The method according to claim 1, wherein the substrate is a planar substrate.

8. The method according to claim 1, wherein the atmosphere is a helium atmosphere.

9. The method according to claim 1, further comprising oxidizing the carbon nano-onions.

10. The method according to claim 1, wherein the pressure is between about 15 pounds per square inch and about 20 pounds per square inch.

11. A functionalized carbon nano-onion comprising a polymeric or an oligomeric functional group bound to the outer shell of the carbon nano-onion.

12. The functionalized carbon nano-onion of claim 11, wherein the functional group is bound to the carbon nano-onion via a carboxyl residue.

13. The functionalized carbon nano-onion of claim 11, wherein the functional group is bound to the carbon nano-onion via a 1-3 dipolar cycloaddition functionalization reaction.

14. The functionalized carbon nano-onion of claim 11, wherein the functional group comprises poly(ethylene glycol).

15. The functionalized carbon nano-onion of claim 11, wherein the functional group comprises a straight-chain hydrocarbon.

16. The functionalized carbon nano-onion of claim 11, wherein the functionalized nano-onion is soluble in an organic solvent.

17. The functionalized carbon nano-onion of claim 11, wherein the functionalized nano-onion is water soluble.

18. The functionalized carbon nano-onion of claim 11, wherein the functional group is bound to the carbon nano-onion via an aryl group.

19. The functionalized carbon nano-onion of claim 11, wherein the functional group is bound to the carbon nano-onion via a 6,6 cyclopropane group.

20. A method for separating a mixture of carbon nano-onions comprising:
providing a mixture of carbon nano-onions, wherein all of the carbon nano-onions are in the mixture in a first physical state; and
reducing a first portion of the carbon nano-onions until the physical state of the first portion of carbon nano-onions is altered to a second physical state, wherein a second portion of the carbon nano-onions remain in the first physical state upon the alteration in state of the first portion.

21. The method according to claim 20, wherein the first physical state is solid and the mixture of carbon nano-onions is provided as a suspension of solids in a solvent.

22. The method according to claim 21, wherein the first portion of the carbon nano-onions is reduced until the first portion dissolves in the solvent, and the second portion remains in suspension upon the dissolution of the first portion.

23. The method according to claim 20 further-comprising functionalizing the carbon nano-onions.

24. The method according to claim 23, wherein the first physical state is a liquid and the mixture of carbon nano-onions is provided as a solution.

25. The method according to claim 24, wherein the second physical state is solid.

26. The method according to claim 25, further comprising depositing the first portion on an electrode.

27. The method according to claim 20, wherein the average size of the carbon nano-onions of the first portion is different than the average size of the carbon nano-onions of the second portion.

28. The method according to claim 20, wherein the reduction is carried out in an electrolytic solution.

29. The method according to claim 20, further comprising oxidizing the mixture of carbon nano-onions.
